(19) United States
(12) Patent Application Publication
(10) Pub. No.: US 2005/0222383 A1
(43) Pub. Date: Oct. 6, 2005

(54) Prostasin substrates and inhibitors

(75) Inventors: Jennifer Harris, San Diego, CA (US); Aaron Shipway, San Mateo, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/051,494

(22) Filed: Feb. 4, 2005

Related U.S. Application Data
(60) Provisional application No. 60/542,163, filed on Feb. 5, 2004.

(51) Int. Cl. C07K 7/08; C07K 7/06

(52) U.S. Cl. 530/324; 530/325; 530/326; 530/327; 530/328; 530/329; 530/330

(57) ABSTRACT

The invention provides substrate specificity profiles for serine protease prostasin. Optimal prostasin substrate sequences, both to the prime side and non-prime side of the prostasin recognition site, are disclosed herein. The prostasin substrate sequences are used in designing substrates, inhibitors, and prodrugs. Prostasin inhibitors based on substrate specificity are also provided.

### CROSS-REFERENCE TO RELATED APPLICATIONS

This application claims the benefit of priority to U.S. Provisional Patent Application Ser. No. 60/542,163 (filed Feb. 5, 2004), the disclosure of which is incorporated herein by reference in its entirety and for all purposes.

### FIELD OF THE INVENTION

The present invention relates to substrate specificity of prostasin and protein substrate design. More particularly, the present invention relates to substrate design for targeting and/or inhibition of prostasin enzyme activity.

### BACKGROUND OF THE INVENTION

Substrate specificity of an enzyme is an important characteristic that governs its biological activity. Characterization of substrate specificity provides invaluable information useful for a complete understanding of often complex biological pathways. In addition, substrate specificity profiles are useful in the design of selective substrates, inhibitors, and prodrugs directed to enzymatic targets. Proteases, also known as proteinases, peptidases, or proteolytic enzymes, are enzymes that degrade proteins by hydrolyzing peptide bonds between amino acid residues. Various categories of proteases include thiol proteases, acid proteases, serine proteases, metalloproteases, cysteine proteases, carboxyl proteases, and the like.

Prostasin (also known as: channel-activating protease; channel-activating protease-1;PRSS8, MERPOPS ID S01.159) is a serine protease of the chymotrypsin-fold. The physiological role of prostasin was unclear until recent studies which functionally identified prostasin as the human homolog to the epithelial sodium channel (ENaC) activating protease in xenopus and mouse (Vallet et al., Nature 389: 607-10, 1997; Adachi et al., J Am Soc Nephrol 12: 1114-21, 2001; and Donaldson et al., J Biol Chem 277: 8338-45, 2002). The epithelial sodium channel (ENaC) controls sodium balance and therefore extracellular airway surface liquid (ASL) volume and blood pressure. Inhibition of prostasin activity provides a potential mechanism of selectively inhibiting ENaC activation in the lung, thereby decreasing sodium hyperabsorption and mucociliary clearance in cystic fibrosis (CF) and possibly other lung disorders such as chronic obstructive pulmonary disease (COPD) and asthma.

Many proteases are non-specific in their activity, e.g., they digest proteins to peptides and/or amino acids. Other proteases are more specific, e.g., cleaving only a particular protein or only between certain predetermined amino acids. Still other proteases have optimal sequences that they cleave preferentially over others. The substrate specificity of prostasin has not been determined, and its availability as a prodrug target has not been previously explored. Improved methods of identifying the optimal substrates of proteases, such as prostasin, are desirable. The present invention fulfills these needs, as well as other needs that will be apparent upon complete review of this disclosure.

### SUMMARY OF THE INVENTION

The present invention provides prostasin substrates, prodrugs, diagnostics and inhibitors, as well as screening and therapeutic methods involving prostasin. In one aspect, the invention provides prostasin-cleavable molecules having a prostasin cleavage site. The prostasin-cleavable molecules typically comprise P₄P₃P₂P₁X, wherein P₁ is arginine; P₂ is tyrosine, leucine, phenylalanine, lysine, asparagine, or valine; P₃ is histidine, arginine, or lysine; P₄ is arginine, lysine, histidine, tyrosine, proline, or leucine; and X comprises one or more of an inhibitory moiety, a label moiety, a polypeptide comprising 1 to 25 amino acids, or a polypeptide that is not attached to P₄P₃P₂P₁ in a naturally occurring protein; and wherein the prostasin cleavage site is between P₁ and X.

In some embodiments, the P₄P₃P₂P₁ in the prostasin-cleavable molecules has a sequence selected from the group consisting of KHYR, RHYR, KHLR, KKLR, KHKR, RKYR, KKYR, KHLR, RHLR, RKLR, and KHKR. In some embodiments of the prostasin-cleavable molecules, X comprises P₁′P₂′P₃′P₄′, wherein P₁′ is attached to P₁ and is histidine, arginine, lysine, asparagine, glutamine, serine, norleucine, or alanine; P₂′ is proline, alanine, histidine, asparagine, norleucine, or glutamine; P₃′ is histidine, serine, glutamine, or aspartic acid; and P₄′ is alanine, serine, norleucine, asparagine, leucine, or threonine. In some embodiments, the label moiety comprises a fluorophore, a coumarin moiety, or a rhodamine moiety.

In another aspect, the invention provides prostasin-cleavable peptides that comprise fewer than 25 amino acids. The peptides comprise P₄P₃P₂P₁, wherein P₁ is arginine or lysine; P₂ is tyrosine, leucine, phenylalanine, lysine, asparagine, or valine; P₃ is histidine, arginine, or lysine; and P₄ is arginine, lysine, histidine, tyrosine, proline, or leucine; and one or more amino acids attached to either or both of P₁ and P₄.

In some of these prostasin-cleavable peptides, P₁ is arginine, P₂ is tyrosine or leucine, P₃ is histidine, lysine, or arginine, and P₄ is lysine or arginine. Some of the peptides further comprise 1 to 20 amino acids linked to P₄. Some of the prostasin-cleavable peptides further comprise 1 to 20 amino acids linked to P₁. Some of the peptides further comprise P₁′P₂′P₃′P₄′, wherein P₁′ is attached to P₁ and is histidine, arginine, lysine, asparagine, glutamine, serine, norleucine, or alanine; P₂′ is proline, alanine, histidine, asparagine, norleucine, or glutamine; P₃′ is histidine, serine, glutamine, or aspartic acid; and P₄′ is alanine, serine, norleucine, asparagine, leucine, or threonine.

In a related aspect, the invention provides prostasin inhibitors. The inhibitors comprise P₄P₃P₂P₁Z, wherein P₁ comprises arginine or lysine; P₂ comprises tyrosine, leucine, phenylalanine, lysine, asparagine, or valine; P₃ comprises histidine, arginine, or lysine; and P₄ comprises arginine, lysine, histidine, tyrosine, proline, or leucine; and Z comprises an inhibitory moiety. The inhibitory moiety can be a transition state analog, a mechanism-based inhibitor, or an electron withdrawing group. Some of the prostasin inhibitors comprise an inhibitory moiety selected from the group consisting of a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide. In some of the prostasin inhibitors, P₁ is arginine, P₂ is tyrosine or leucine, P₃ is histidine, lysine, or arginine, and P₄ is lysine or arginine. In some inhibitors, P₄ comprises acetyl-lysine.

In one aspect, methods for identifying a modulator of prostasin are provided. The methods involve the steps of (a) contacting a test agent with prostasin in the presence of a prostasin substrate of the invention, and (b) detecting an alteration of cleavage of the prostasin substrate by prostasin in the presence of the test agent relative to cleavage of the prostasin substrate by prostasin in the absence of the test agent; thereby identifying a prostasin modulator.

In another aspect, the invention provides methods for reducing a prostasin activity in a cell. The methods entail contacting the cell with a prostasin inhibitor molecule of the invention, thereby reducing prostasin activity in the cell.

### BRIEF DESCRIPTION OF THE DRAWINGS

FIGS. 1A-1C show results of positional scanning of a putative prostasin substrate library of two-position fixed tetrapeptide for the non-prime side.

FIGS. 2A-2D show results of kinetic assays of prostasin activity on putative prostasin substrate libraries of peptides with one-position fixed prime side sequence and an optimal non-prime side sequence.

FIG. 3 shows pH profiling of prostasin activity.

### DETAILED DESCRIPTION

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a prostasin substrate” includes a combination of two or more substrates; reference to “bacteria” includes mixtures of bacteria, and the like.

In some embodiments of the present invention, active prostasin is optionally expressed in an E. coli, baculovirus, or other available expression system and can be used to generate a substrate specificity profile, e.g., a profile comprising primary and extended specificity on the prime and/or non-prime sides of the cleavage site of prostasin. For example, positional scanning formats are optionally used with tetrapeptide libraries of putative substrates to provide a substrate profile. Substrates are identified, synthesized and tested for prostasin cleavage. In addition, the substrate profile is optionally used to develop prostasin inhibitors and prodrugs, e.g., compositions that can be selectively activated (e.g., cleaved and released) where they can inhibit the enzymatic activity of prostasin, thereby inhibiting ENaC activation. Furthermore, the specificity information can optionally be used to identify or confirm physiological substrates and biological pathways in which prostasin operates.

In one embodiment of the present invention, the substrate specificity information obtained for prostasin is optionally used to design sequences into small molecule substrates using fluorescence resonance energy transfer or other fluorescent or chromagenic signals to observe prostasin activity in vitro, ex vivo, or in vivo. In another embodiment, the sequences are optionally designed into a prodrug format in which the drug is only activated and/or released at sites where prostasin is expressed (e.g., the lung).

I. Prostasin and Prostasin Polypeptides

A typical enzyme of interest in the present invention is prostasin, a serine protease. “Protease,” as used herein, typically refers to an enzyme that degrades proteins or peptides, e.g., by hydrolyzing peptide bonds between amino acid residues. Prostasin, also known as channel-activating protease; channel-activating protease-1; PRSS8, MERPOPS ID S01.159, is a serine protease of the chymotrypsin-fold. Prostasin was first isolated from seminal fluid (Yu et al., J Biol Chem 269: 18843-8, 1994). mRNA expression profiling shows that it has a widespread tissue distribution—with significant expression in the pancreas, thyroid, prostate, salivary gland, trachea, lung, kidney and liver. Prostasin is synthesized as a pre-pro-enzyme of 343 amino acids with a possible C-terminal transmembrane (or GPI consensus) domain that suggests that it may be membrane associated with the catalytic domain on the extracellular or luminal surface (Chen et al., J Biol Chem 276: 21434-42, 2001).

In the present invention, the term “prostasin” is used to refer to any portion of the prostasin protease which exhibits substantially similar cleavage patterns to an intact prostasin molecule. For example, a prostasin molecule typically comprises a transmembrane domain, a pro-domain, and a catalytic domain. However, for many screening applications, a soluble form of prostasin, e.g., without the transmembrane domain, is preferred.

As applied to prostasin, the terms “polypeptide” and “protein” are used interchangeably. Prostasin polypeptides of the invention include, but are not limited to, proteins, biotinylated proteins, isolated proteins, and recombinant proteins. In addition, the polypeptides or proteins of the invention optionally include naturally occurring amino acids as well as amino acid analogs and/or mimetics of naturally occurring amino acids, e.g., that function in a manner similar to naturally occurring amino acids. In the present invention, prostasin polypeptides or peptides can also optionally contain amino acids analogs, derivatives, isomers (e.g., L or D forms of the amino acids), and/or conservative substitutions of amino acid residues. A conservative substitution refers to the replacement of one amino acid with a chemically-similar residue, e.g., the substitution of one hydrophobic residue for another. Exemplary substitutions include, but are not limited to, substituting alanine, threonine, and serine for each other, asparagine for glutamine, arginine for lysine, and the like.

II. Substrate Libraries for Profiling Prostasin Substrate Specificity

For many screening applications, e.g., screens for prostasin activity or prostasin substrate specificity profiles, a library of substrates or putative substrates is desired. A “library” is a collection or group of molecules, e.g., about 350-400 or more molecules, about 1000 or more molecules, about 10,000 or more, and/or about 100,000 or more molecules. Typically, each member of the library comprises a different molecule. As such, the number of members in a given library of the present invention is optionally the number of constitutive components, or substrate moiety options (e.g., 19-20 amino acid options), to the power of how many positions are being varied (e.g., 3 positions in a 1-fixed-position tetrapeptide). For example, a library of tetrapeptide substrates generated using 20 amino acids and keeping the P₁ position fixed as arginine can comprise a maximum collection of (20)³ or 8,000 different peptide sequences that are potentially cleavable by prostasin.

A library of putative prostasin substrates is a library or collection of molecules that may or may not be cleavable by prostasin. It can be created using peptide synthesis techniques well known to those of skill in the art, or the techniques described in PCT application WO 03/029823 (“Combinatorial Protease Substrate Libraries”). Such a library is used, e.g., to probe substrate specificity. These libraries are optionally used to provide non-prime side information regarding the enzyme active site with respect to the various member substrates of the library. For example, an optimal non-prime substrate sequence, e.g., the first four amino acids on the non-prime side (e.g., N-terminal side) of the cleavage site can be identified for prostasin. This information is optionally used to design more selective and/or potent substrates. For example, different fluorogenic compounds are optionally employed to increase the sensitivity (e.g., detection sensitivity) of these substrates. The substrates identified also can provide valuable diagnostics for the identification of protease activity in complex biological samples, and are valuable in screening efforts to identify protease inhibitors.

Members of the substrate libraries or putative substrate libraries typically comprise from about 1 to about 15 substrate moieties, or from about 4 to about 25 substrate moieties. The term “substrate moiety” refers to a component of the substrate molecule, and as such includes any amino acid or amino acid mimetic, as well as the labels, therapeutic molecules, inhibitory molecules described herein, and other components of interest. In addition, selected components are optionally coupled to or linked to the substrates. Such selected components include, but are not limited to: peptides, proteins, non-peptide moieties, sugars, polysaccharides, polyethylene glycol, small molecules, organic molecules, inorganic moieties, label moieties, therapeutic moieties, and/or the like.

Typically, the substrate moieties and selected components, when used in a substrate or putative substrate, form a prostasin cleavage site or a potential prostasin cleavage side, e.g., prostasin cleaves between two of the substrate moieties, such as between two amino acids or between an amino acid and a coumarin moiety. In some embodiments, the substrate moieties comprise amino acids which provide prime side and/or non-prime side specificity to a prostasin cleavage site. In other embodiments, labels that allow for detection of a cleavage event are incorporated into the substrates of the invention.

Members of the substrate libraries can further comprise a label moiety. The label moiety can be a molecule with fluorescent properties which alter upon cleavage from the substrate, or a matched donor:acceptor pair of fluorescence resonance energy transfer (FRET) compounds. In one embodiment, a fluorescence donor moiety and a fluorescence acceptor moiety are attached to the putative prostasin substrate library members on opposite sides of the putative prostasin cleavage site, such that monitoring the cleavage of the putative prostasin substrates is performed by detecting a fluorescence resonance energy transfer. Monitoring can include detecting a shift in the excitation and/or emission maxima of the fluorescence acceptor moiety, which shift results from release of the fluorescence acceptor moiety from the putative prostasin substrate by the prostasin activity.

In some embodiments, members of the prostasin substrate libraries or putative substrate libraries have one or more positions in the peptide sequence held constant while the others are varied. These libraries, also known as positional scanning libraries, can be created to probe the prime and/or non-prime specificity of prostasin. As one example, four 20-well sub-libraries can be optionally created, wherein each of the four sub-libraries has a different fixed amino acid position, e.g., P₁, P₂, P₃, or P₄. For example, in a first sub-library, each of the twenty wells contains a library of substrates wherein P₁ is fixed at one of twenty different amino acids, while the other positions, P₂, P₃, and P₄, are varied. In some embodiments of the present invention, the libraries contain about 6859 different substrates per well (i.e., one fixed position and three variable positions per substrate, and using 19 different amino acids during generation of the library, cysteine having been excluded from the synthesis mixture).

Additional sub-libraries can also be optionally created, e.g., with two fixed positions, e.g., P₁/P₂, P₁/P₃, P₁/P₄, P₂/P₃, P₂/P₄, or P₃/P₄. This produces six sub-libraries of 400 wells each (representing each possible combination of the two fixed elements, and the 20 possible elements in each of the fixed positions), wherein each well contains about 361 different substrate sequences (e.g., using the 19 amino acids in the two variable positions). Therefore, the libraries of the invention typically involve about 2400 wells total and the libraries contain well over 100,000 different substrates, e.g., coumarin based substrates. The preferred amino acid for each position, e.g., in a prostasin substrate, is optionally determined using these positional scanning libraries. See, e.g., Harris et al. (2000) Proc. Natl. Acad. Sci USA 97:7754-7759 for a general description of how such libraries are used to determine optimal substrate sequences.

A non-prime side positional scanning library is typically constructed using a detectable moiety, e.g., a moiety that is not detectable until after it has been cleaved from the substrate (e.g., the peptide). For example, members of a non-prime side scanning library can comprise P₄P₃P₂P₁X, wherein P₄-P₁ comprise amino acids or amino acid mimetics randomized as described above and X comprises a detectable moiety, such as coumarin.

Optionally, prime side specificity can also be analyzed or probed using putative substrate libraries of the present invention. In a preferred embodiment, a prime side position library, e.g., for determining prime side substrate specificity, is constructed using a donor moiety, an acceptor moiety, and a preselected non-prime substrate sequence. Donor moieties and acceptor moieties in the present invention can comprise fluorescence resonance energy transfer pairs. A typical donor moiety for use in the present invention absorbs light at one wavelength and emits at another wavelength, typically a higher wavelength. The acceptor moiety of the invention typically absorbs at the wavelength of either the absorption or emission wavelength of the donor moiety. For example, the acceptor is used as a quencher for the donor moiety. However, the acceptor typically only quenches the absorption or emission of the donor when the two are in proximity, either in high concentrations or when tethered to each other, e.g., chemically bonded. The donor-acceptor pairs are then used to detect protease cleavage, e.g., prostasin cleavage, of the substrates of the libraries in the present invention. For example, when cleavage occurs, the acceptor no longer quenches the signal of the donor.

One or more prime position substrate moiety is typically coupled to an acceptor moiety. The prime substrate moieties typically comprise amino acids or amino acid mimetics which are used to form a prostasin cleavable molecule. In a typical library, about four substrate moieties are coupled to the acceptor, e.g., P₁′, P₂′, P₃′, and P₄′. However, the number of substrate moieties coupled to the acceptor is optionally varied, e.g., from about 1 to about 15, but is more typically, about 2 to about 6, and most typically four. Typically, the substrate moieties are coupled to an acceptor using standard peptide synthesis techniques, e.g., Fmoc synthesis.

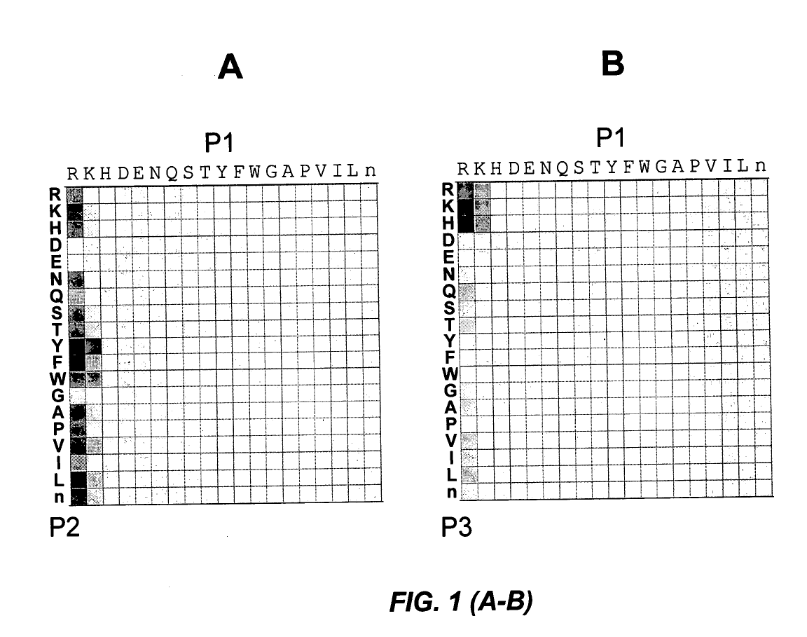

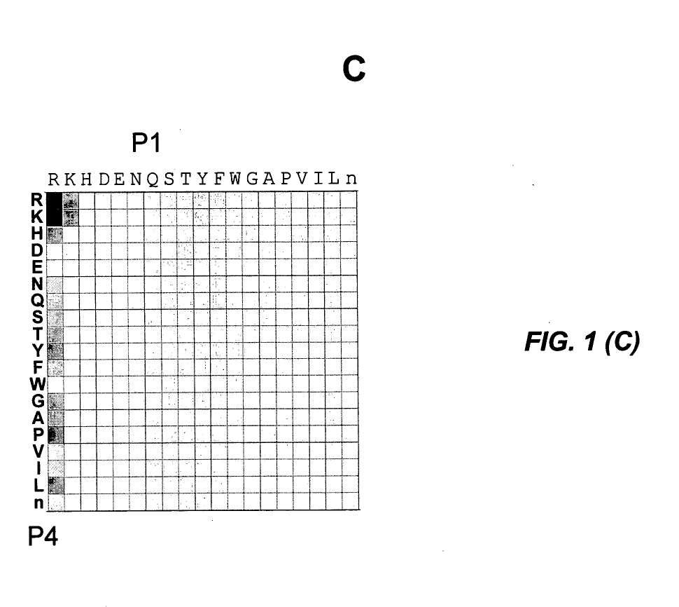

After the prime side positional substrate is coupled to the acceptor, a preselected non-prime substrate, e.g., an optimal or preferred non-prime sequence that has been identified, is coupled to the prime position substrate. “Preselected substrate moieties” are determined as described above and in PCT application WO 03/029823, using, e.g., a positional scanning library. The preselected sequences are typically about 2 to about 20 substrate moieties, e.g., amino acids, in length, more typically about 2 to about 6, and most typically about 4 amino acids or substrate moieties in length. As shown in FIG. 1, preselected non-prime side substrate sequences for prostasin (P₄P₃P₂P₁) could include, e.g., the tetrapeptides KHYR, RHYR, KHLR, KKLR, KHKR, RHFR, RKYR, KKYR, KHLR, RHLR, RKLR, KHKR, and RHFR.

III. Profiling Prostasin Substrate Specificity

The invention provides methods for screening substrate library and profiling substrate specificity of prostasin. To profile prostasin substrate specificity, a library of prostasin substrates as described above (e.g., a coumarin-based substrate library) is provided. Each member of the library comprises a putative prostasin recognition site. The substrate profile is obtained by monitoring cleavage of the substrates by prostasin. Often, to obtain a complete substrate profile for an enzyme, e.g., a protease, a non-prime scan and a prime scan are performed. A “non-prime scan” refers to the scanning library used to determine an optimal substrate sequence for the non-prime side of the cleavage site and/or the results of an analysis of that library. A “prime side scan” refers to the opposite side of the cleavage site, either the library used to probe those positions or the results of such a probe.

Typically, an optimal substrate sequence for the non-prime positions is determined first, using techniques known in the art (e.g., non-prime side scan as exemplified in the Examples below). Thereafter, a second substrate library (e.g., a prime side scan library) is prepared. In some embodiments, a library for a prime scan (e.g., a library for probing prime side substrate sequence specificity) can be prepared using a fluorescence donor-acceptor pair and the optimal non-prime sequences obtained, e.g., as described above. The prime side scan library is then incubated with the enzyme of interest and monitored to determine one or more optimal prime substrate sequence.

As noted above, the substrate moieties that occupy one or more of the non-prime positions can be preselected to allow cleavage of the substrate at the putative prostasin cleavage site by the prostasin, while allowing the moieties on the prime-side of the cleavage site to vary. Alternatively, both the substrate moieties that occupy the non-prime and the prime positions vary among different members of the library of prostasin substrates (e.g., no pre-selection of library members).

FIG. 1 provides data obtained from incubating a non-prime scan library of coumarin-based substrates with prostasin. The figure depicts the enzyme activity for pools of library members having two “fixed” positions in the tetrapeptide-coumarin substrate. When prostasin acts on a substrate, the substrate is cleaved between P₁ and the coumarin moiety, thereby releasing a fluorogenic coumarin moiety, which is detected. As shown in the figure, arginine is the most preferred P₁ residue. Preferred residues for positions P₂-P₄, based on having a fixed P₁ substituent, are also illustrated in the figure. For example, the preference in the P₂ position is tyrosine, leucine, phenylalanine, lysine, asparagine, or valine. The P₃ position prefers histidine, arginine, or lysine. The P₄ position also prefers arginine, lysine, histidine, as well as tyrosine, proline, and leucine.

After the non-prime side sequence is determined, a second library can be constructed to determine the prime side substrate specificity of prostasin. The non-prime side sequence of members of this second substrate library sequence is preselected based on the information obtained from the non-prime scan. For example, the non-prime side of the substrate in the second substrate library of the invention can be kept constant as the sequence determined from a coumarin library, P₄-Lys, P₃-His, P₂-Tyr, P₁-Arg, or any other sequence as provided above. The prime-side four amino acid positions are typically randomized as all 20 natural amino acids. However, in some embodiments, norleucine is optionally used to replace methionine and/or cysteine is optionally excluded.

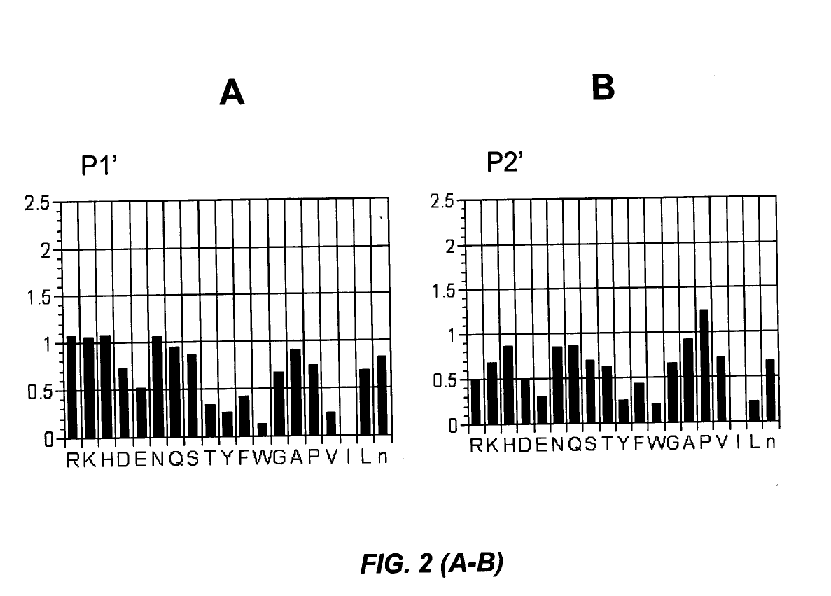

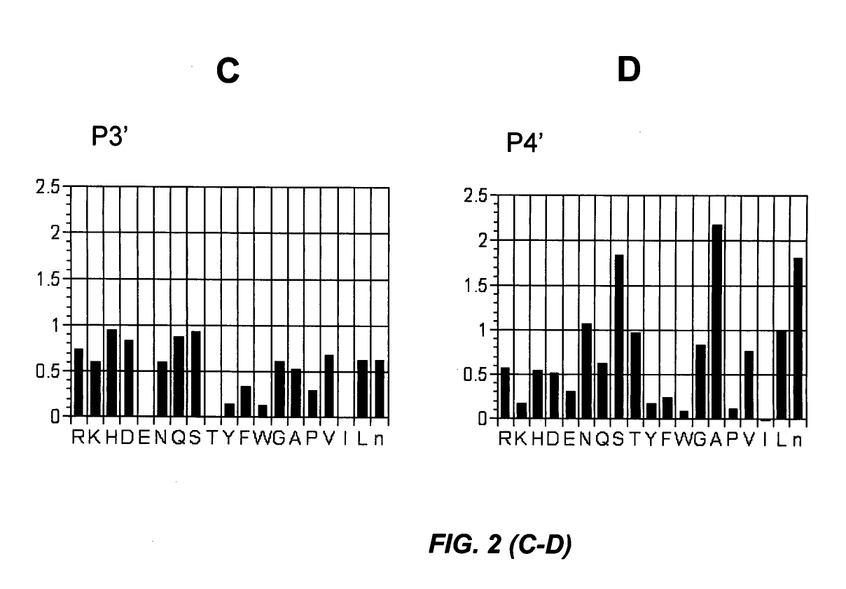

An exemplary specificity profile, with the preselected non-prime side sequence being P₄-Lys, P₃-His, P₂-Tyr, P₁-Arg, is provided in FIG. 2. Panels A-D provide prime side substrate specificity for P₁′, P₂′, P₃′ and P₄′ with the y-axis representing relative fluorescence units per second and the x-axis representing the amino acid held constant in the substrate. The results indicate that the preferred residues for the prime side sequence are P₁′: histidine, arginine, lysine, asparagine, glutamine, serine, norleucine, or alanine; P₂′: proline, alanine, histidine, asparagine, norleucine, or glutamine; P₃′: histidine, serine, glutamine, or aspartic acid; and P₄′: alanine, serine, norleucine, asparagine, leucine, or threonine.

The prime and non-prime side sequence of prostasin substrate as determined above can be used to search genomic databases, e.g., for similar cleavage sites in proteins and provide possible macromolecular substrates that are key to the biological function of prostasin. In addition, the information is useful to design peptide based inhibitors of prostasin and prodrugs and diagnostic reagents based on prostasin specificity.

The prime and non-prime information can also be used to design more selective and potent substrates, e.g., for use as therapeutic agents or biological tools. Multiple fluorogenic compounds can be employed with the determined amino acid specificity sequence to increase the sensitivity and efficacy of these substrates for a particular system.

Furthermore, substrates of the present invention are valuable as diagnostics for the identification of protease activity in complex biological samples and for screening efforts to identify protease inhibitors. The overall strategy when applied, e.g., to an entire class of proteases, provides panning information that allows for the generation of specific substrates and inhibitors in the context of an entire protease class. The non-prime and prime specificity information can be employed to bias bead-based and phage display methods, to design cleavage sites in fusion proteins or other protein constructs, and to design prodrugs in which the protease target releases an active drug. These are described in more detail below.

IV. Prostasin-Cleavable Substrates

In one aspect, the invention provides prostasin-cleavable substrates. Typically, such prostasin substrates are peptide-based molecules that are cleavable by prostasin, including protein, polypeptide and peptide substrates. The substrates also include non-peptide substrates and substrates comprising a peptide attached to a non-peptide moiety. The prostasin recognition sites employed in the present invention typically comprises an amino acid sequence, e.g., about 4 to about 25 amino acids. The amino acids are typically selected to form a prostasin specific cleavage site, e.g., a sequence that is cleavable by prostasin. In addition, the sequence is preferably specific for prostasin, e.g., it is not cleaved by other proteases. The recognition site is typically a portion of a prostasin substrate, which is cleaved by prostasin upon recognition. For example, a recognition site typically comprises one or more residue to which prostasin binds prior to cleavage. Cleavage yields can range anywhere from about 0.1% to 100% cleavage of the substrate.

In some embodiments of the present invention, the prostasin substrates comprise Pₙ . . . P₄P₃P₂P₁P₁′P₂′P₃′P₄′ . . . Pₙ′. As used herein, the nomenclature for substrates refers to prime side and non-prime side positions, wherein each Pₙ and Pₙ′ (alternatively referred to as P₋ₙ) is typically a substrate component or moiety, such as an amino acid or amino acid mimetic. Cleavage, e.g., amide bond hydrolysis, typically occurs between P₁ and P₁′ (see, e.g., Schechter and Berger (1968) Biochem. Biophys Res. Commun. 27:157-62). For example, prostasin typically cleaves an amide bond between two substrate moieties, such as between an amino acid in a prime side peptide P₁ position and an amino acid in a non-prime side peptide P₁′ position. Optionally, “n” ranges from zero to 21 substrate moieties, thereby providing substrates with various number of units (e.g., amino acids) in length.

In other embodiments, the substrates comprise Pₙ . . . P₄P₃P₂P₁X, wherein X is a selected component such as a peptide, a protein, a label moiety, a therapeutic moiety, or the like. For example, in some embodiments, prostasin cleaves a substrate between P₁ and X, wherein P₁ is a peptide moiety (e.g. an amino acid), and X is a diagnostic moiety such as a coumarin compound which fluoresces upon release from the peptide. In some embodiments, the N-terminal amino acid of the substrate is protected, e.g., by acetylation. Other N-terminal protecting groups such as like Z, Cbz, or succinate can also be employed in the prostasin substrates of the invention.

A peptide or substrate of the invention is “cleavable by” prostasin if, when mixed with a prostasin molecule, the substrate or peptide is cleaved, e.g., at a cleavage site as described above, e.g., between the P₁ and P₁′ positions or between P₁ and X. The prostasin substrates of the invention typically comprises a non-prime side sequence (e.g., to the N-terminal side of the cleavage site) and an additional moiety, e.g., a prime side sequence (e.g., to the C-terminal side of the cleavage site), a therapeutic moiety, or a diagnostic moiety (e.g., a fluorophore). When a substrate molecule is cleaved by prostasin, the additional moiety is released from the peptide upon cleavage, unless the additional moiety is coupled to the substrate molecule at a second position distal from the cleavage site.

Some of the prostasin substrates of the present comprise a tetrapeptide sequences in which P₁ is arginine; P₂ is tyrosine, leucine, phenylalanine, lysine, asparagine, or valine; P₃ is histidine, arginine, or lysine; and P₄ is arginine, lysine, histidine, tyrosine, proline, or leucine. Optionally, the amino group of the N-terminal amino acid (e.g., P₄) is derivatized or blocked. Preferably, the N-terminal amino acid of the tetrapeptide is N-acetylated. Preferably P₄ is selected from the group consisting of arginine, lysine, histidine, tyrosine, proline, leucine, glycine, and alanine. Preferred peptides for use in the prostasin-cleavable molecules of the present invention include KHYR, RHYR, KHLR, KKLR, KHKR, RHFR, RKYR, KKYR, KHLR, RHLR, RKLR, KHKR, and RHFR, as illustrated in FIG. 1.

In addition to the above described peptide sequences, the prostasin cleavable molecules of the present invention can comprise an additional component X, wherein X comprises a therapeutic moiety, a label moiety, a polypeptide (e.g., comprising from about 1 to about 25 amino acids, such as the a prime-side coupled peptides described herein), or a non-native or non-naturally occurring peptide sequence, e.g., one not found in a naturally-occurring prostasin substrate. Other X components that are optionally included in the prostasin substrates of the invention include, but are not limited to: polyalcohols such as polyethylene glycol, biotin, various carbohydrates or carbohydrate polymers, or crosslinking agents. The X component can be coupled or attached to the prostasin cleavable molecule at either or both of the P₁ and P₄ moieties. In some embodiments, the prostasin cleavable molecules are provided in the format P₄P₃P₂P₁X and are cleavable by prostasin between the P₁ moiety of the peptide sequence and the X component.

In some embodiments, component X comprises a prime-side peptide or peptide-like sequence (the units of which are designated Pₙ′, or sometimes P₋ₙ). For example, a prostasin cleavable molecule or prostasin substrate of the invention optionally comprises a non-prime side sequence and a prime side sequence as described above (e.g. Pₙ . . . P₄P₃P₂P₁P₁′P₂′P₃′P₄′ . . . Pₙ). Preferred prime sequences are those in which P₁′ is histidine, arginine, lysine, asparagine, glutamine, serine, norleucine, or alanine; P₂′ is proline, alanine, histidine, asparagine, norleucine, or glutamine; P₃′ is histidine, serine, glutamine, or aspartic acid; and P₄′ is alanine, serine, norleucine, asparagine, leucine, or threonine, as illustrated in FIG. 2.

Once a prostasin substrate sequence is determined, e.g., from a positional scanning library as described below or by other methods known in the art, the substrate peptides of the present invention are typically synthesized using any recognized procedure in the art, e.g., solid phase synthesis, e.g., t-boc or fmoc protection methods, which involve stepwise synthesis in which a single amino acids is added in each step starting with the C-terminus. See, e.g., Fmoc Solid Phase Peptide Synthesis: A Practical Approach in the Practical Approach Series, by Chan and White (Eds.), 2000 Oxford University Press. The peptides are then optionally used to provide substrates, inhibitors, prodrugs, diagnostics, etc. as described below.

In forming the various prodrugs, diagnostics, inhibitors, and the like, the peptide sequences provided herein are optionally linked to non-peptide moieties, e.g., aldehydes, cytotoxic compounds, labels, or other additional components. As detailed below, such non-peptide moieties are typically coupled to the peptide sequences, either directly, e.g., via a covalent bond (such as an amide bond or carbamate linkage), or indirectly via a linker molecule (such as a glycol linker or Rink linkers).

V. Prostasin Substrate Based Prodrugs

A “prodrug” is a composition that is modified to become active, often in vivo. Such compositions typically comprise a therapeutic moiety or cell modulating moiety that is cleaved from the remainder of the composition, preferably at a target site. The therapeutic or cell-modulating moiety is typically activated only after cleavage from the remainder of the composition. The prodrugs of the invention are typically peptides linked to therapeutic moieties. The therapeutic moieties can be linked to the prostasin substrate peptides either directly or indirectly, e.g., via a covalent bond, or a spacer or linker molecule. The attachment or linkage of the therapeutic moiety to the peptide moiety of the invention typically results in limiting the function of the moiety while attached to the peptide. The moiety is then activated or available for use after being cleaved from the peptide. Therefore, the prodrugs of the invention are not generally toxic. For example, a therapeutic moiety has an affect only when cleaved, e.g., in the presence of prostasin.

A “therapeutic moiety” of the invention is a compound, molecule, substituent, or the like, that relates to the treatment or prevention of a disease or disorder, e.g., to provide a cure, assist in a cure or partial cure, or reduce a symptom of the disease or disorder. In the present invention, therapeutic moieties are typically linked to the carboxyl terminus of the peptides of the invention, e.g., at P₁. The therapeutic moiety or drug is optionally linked directly to the peptide or via a linker. Direct linkage typically involves an amide bond or an ester bond. When a linker is used, any type of linkage or bond known to those of skill in the art is optionally used.

When a linker is used to attach the therapeutic moiety to the peptide portion of the prodrug, the linker is optionally cleaved from the peptide moiety along with the therapeutic moiety, or it remains attached. If the linker remains with the therapeutic moiety after cleavage by prostasin, it does not typically affect the function or toxicity of the therapeutic moiety. In other embodiments, the linker or spacer group is self-cleaving. Self-cleaving or self-immolative linkers are those designed to cleave or spontaneously eliminate from the therapeutic moiety after cleavage of the therapeutic moiety from the peptide. For information on self-cleaving linkers useful in prodrugs, see, for example, U.S. Pat. No. 6,265,540 B1.

When administered to a subject, the prodrugs of the invention are typically provided in an aqueous or non-aqueous solution, suspension, or emulsion. Suitable solvents are known to those of skill in the art and include, but are not limited to, polyethylene glycol, ethyl oleate, water, saline, and the like. Preservatives, and other additives are also optionally included, e.g., antimicrobials.

VI. Prostasin Substrate Based Diagnostics

In addition to prodrugs, the prostasin substrates of the present invention are also used as diagnostic reagents or components thereof. For example, a prostasin substrate of the invention is optionally linked to a fluorescent molecule, e.g., one that fluoresces only after cleavage from the substrate, to provide a diagnostic moiety that is used to detect the presence of prostasin or in high throughput screening of prostasin inhibitors.

A “diagnostic moiety” is a compound, molecule, substituent, or the like, that is used, e.g., to distinguish or identify, e.g., a certain disease, condition, or diagnosis. For example, the presence of prostasin is an example of a condition that a diagnostic of the invention is optionally used to identify. A diagnostic moiety of the invention is typically a label moiety that fluoresces upon cleavage from a prostasin substrate and allows the detection of the cleavage event, e.g., that is used to detect the presence of prostasin.

A “label moiety” is any detectable compound, molecule, or the like. When attached to a prostasin substrate of the invention, the labels provide for detection of prostasin. Typically, the labels of the present invention do not become detectable until after a cleavage event has occurred, e.g., cleaving the label from a prostasin substrate. A label is detectable by any of a number of means, such as fluorescence, phosphorescence, absorbance, luminescence, chemiluminescence, radioactivity, colorimetry, magnetic resonance, or the like.

Label moieties of the invention include, but are not limited to, absorbent, fluorescent, or luminescent label moieties. Exemplary label moieties include fluorophores, rhodamine moieties, and coumarin moieties (e.g., such as 7-amino-4-carbamoylcoumarin, 7-amino-3-carbamoylmethyl-4-methylcoumarin, or 7-amino-4-methylcoumarin). Typically, a label moiety exhibits significantly less absorbance, fluorescence or luminescence when attached to the prostasin-cleavable molecule than when released from the prostasin-cleavable molecule. For example, a fluorophore emits light when it is exposed to the wavelength of light at which it fluoresces. The emitted light is detected. In the present invention, fluorophores with attenuated fluorescence until separated from the attached peptide are typically used. Therefore, a prostasin substrate with an attached fluorophore has attenuated fluorescence or provides a diminished signal until the substrate is cleaved by prostasin, thereby releasing the fluorophore. In this manner, the presence of prostasin is easily detected using the substrates of the invention. Fluorophores of interest include, but are not limited to, fluorescein, fluorescein analogs, BODIPY-fluorescein, arginine, rhodamine-B, rhodamine-A, rhodamine derivatives, green fluorescent protein (GFP), and the like. For further information on fluorescent label moieties and fluorescence techniques, see, e.g., Handbook of Fluorescent Probes and Research Chemicals, by Richard P. Haugland, Sixth Edition, Molecular Probes, (1996).

An exemplary label moiety that does not fluoresce until cleaved from the substrate is a coumarin moiety. A “coumarin moiety” is a compound or molecule comprising a coumarin compound. Coumarin compounds of interest in the present invention include, but are not limited to, 7-amino-4-carbamoylmethylcoumarin (“acc”), 7-amino-4-methylcoumarin (“amc”), 7-methoxy-4-carbamoylmethylcoumarin, and 7-dimethylamino-4-carbamoylmethylcoumarin, and the like. Many other coumarin compounds are available, e.g., either commercially (see, e.g., Sigma and Molecular Probes catalogs) or using various synthetic protocols known to those of skill in the art. The synthesis of an exemplary coumarin compound of interest is described in WO 03/029823.

The substrates linked to a coumarin moiety can have the non-prime and/or prime side amino acid sequences as provided above. For basic strategies for preparation of and use of coumarin-based substrates and coumarin libraries, see, e.g., Zimmerman et al. (1977) Analytical Biochemistry 78:47-51; Lee et al. (1999) Bioorganic and Medicinal Chemistry Letters 9:1667-72; Rano et al., supra; Schechter and Berger (1968) Biochemical and Biophysical Chemistry Communications 27:157-162; Backes et al. (2000) Nature Biotechnology 18:187-193; Harris et al. (2000) “Rapid and general profiling of protease specificity by using combinatorial fluorogenic substrate libraries” Proc. Natl. Acad. Sci USA 97:7754-7759; and Smith et al. (1980) Thrombosis Res. 17:393-402.

In other embodiments, quantum dots are optionally used as diagnostic moieties. Nanocrystals, e.g., semiconductor nanocrystals or quantum dots such as cadmium selenide and cadmium sulfide, are optionally used as fluorescent probes. Quantum dots typically emit light in multiple colors, which allows them to be used to label and detect several compounds or samples at once. See, e.g., Bruchez et al. “Semiconductor Nanocrystals as Fluorescent Biological Labels,” Science 281:2013-2016 (1998). Quantum dot probes are available, e.g., from Quantum Dot Corporation (Hayward, Calif.).

In the present invention, a quantum dot is optionally linked to or associated with a prostasin substrate and used to detect the substrate, e.g., after cleavage by prostasin. In some embodiments, the label moiety optionally comprises a first quantum dot attached to a prostasin cleavable molecule on one side of the prostasin cleavage site and a second quantum dot attached to the molecule on the opposite side of the prostasin cleavage site. Typically, the first and second quantum dots emit signals of different wavelengths upon illumination. For example, a quantum dot is optionally linked to a prime side of a peptide substrate as described above, e.g., using standard chemistry techniques, and a differently colored quantum dot is linked to the non-prime side of the substrate. Detection of the quantum dots allows detection of a cleavage event when the prime and non-prime sides are cleaved from each other, e.g., by prostasin.

Alternatively, electroactive species, useful for electrochemical detection, or chemiluminescent moieties, useful for chemiluminescent detection, are incorporated into the prostasin substrates or putative substrates of the invention. UV absorption is also an optional detection method, for which UV absorbers are optionally used. Phosphorescent, colorimetric, e.g., dyes, and radioactive labels are also optionally attached to the prostasin substrates of the invention, e.g., using techniques well known to those of skill in the art.

Labels as described above are typically linked to the prostasin substrates of the invention using techniques well known to those of skill in the art. For example, the label or diagnostic moiety is typically linked to P₁ as P₄P₃P₂P₁X, wherein X comprises the label moiety. Alternatively, the label moiety is linked to the prime side of a prostasin substrate or to P₄. In some embodiments, the label moiety comprises two labels, such as two quantum dots. One label is attached to the prime side of the substrate and the other label is attached to the non-prime side of the substrate, as ′X₁P₄P₃P₂P₁P₁′P₂′P₃′P₄′X₁′, wherein X₁ and X₁′ each comprise a label moiety, such as quantum dot or a member of a FRET pair. In other embodiments, the label moiety is optionally attached to any of the substrate moieties, e.g., P₄-P₁, or P₁′-P₄′. P₄-P₁ and P₁′-P₄′ can be the amino acid sequences as described above for the non-prime and prime sides, respectively, of prostasin recognition site.

The present invention also provides methods of labeling a cell using the labeled prostasin-cleavable molecules of the present invention. The labeling method include contacting the cell with a prostasin-cleavable molecule that comprises a prostasin cleavage site, wherein the prostasin-cleavable molecule comprises the structure P₄P₃P₂P₁X, wherein the prostasin cleavage site is between P₁ and X; and wherein P₁ is arginine; P₂ is tyrosine, leucine, phenylalanine, lysine, asparagine, or valine; P₃ is histidine, arginine, or lysine; and P₄ is arginine, lysine, histidine, tyrosine, proline, or leucine; and X comprises a label moiety. A variety of labels can be incorporated into the prostasin-cleavable molecules of the present invention, including, but not limited to, a coumarin moiety and members of a donor-acceptor FRET pair, as described herein.

In a further aspect, the present invention provides methods of screening a subject for increased prostasin activity or expression. First, a cell or tissue sample is obtained from the individual. The cell or tissue sample is then put into contact with one or more prostasin-cleavable molecules that comprise a prostasin cleavage site and a detectable label moiety. The prostasin-cleavable molecules can comprise P₄P₃P₂P₁X, wherein the prostasin cleavage site is between P₁ and X; and wherein P₁ is arginine; P₂ is tyrosine, leucine, phenylalanine, lysine, asparagine, or valine; P₃ is histidine, arginine, or lysine; and P₄ is arginine, lysine, histidine, tyrosine, proline, or leucine; and wherein X comprises a label moiety. Any prostasin activity in the sample can be monitored by detecting a signal of the label moiety from the prostasin cleavable molecule. The level of detected label is compared to a control or standard level of prostasin activity, thereby determining whether the prostasin activity or expression is increased.

VII. Prostasin Substrate Based Prostasin Inhibitors

The invention also provides prostasin inhibitors. Enzyme inhibitors are typically compounds or molecules that negatively affect the ability of an enzyme to catalyze a reaction. A “prostasin inhibitor” is a protease inhibitor that inhibits, curbs, or decreases the activity of prostasin. A typical prostasin inhibitor of the present invention, P₄P₃P₂P₁Z, comprises a prostasin recognition site such as a peptide sequence P₄P₃P₂P₁ as described above. The peptide sequence is typically linked to an inhibitory moiety, Z. An “inhibitory moiety” is a compound or chemical group that is capable of inhibiting prostasin activity when associated with the prostasin protease, such as a transition state analog, a mechanism-based inhibitor, an electron withdrawing group, a chemical modifier, or the like. Exemplary inhibitory moieties include, but are not limited to, a C-terminal aldehyde, a boronate, a phosphonate, an α-ketoamide, a chloro methyl ketone, a sulfonyl chloride, ethyl propenoate, vinyl amide, vinyl sulfone, or vinyl sulfonamide.

Mechanism-based inhibitors for various catalytic reactions are well known, e.g., synthetase, peptidase, oxidation/reduction, β-lactamase, decarboxylation, aminotransferase, lyase, racemase, and hydroxylase reactions (Silverman, Chemistry and Enzymology, Vols. I and II, CRC Press, 1988, Boca Raton, Fla.). One of skill in the art can similarly design mechanism based inhibitors for prostasin based on the prior art disclosure (see, e.g., Silverman, Chemistry and Enzymology, Vols. I and II, CRC Press, 1988, Boca Raton, Fla.; and U.S. Pat. No. 6,177,270). Electron withdrawing groups are also well known in the art. For example, they include chemical groups such as halogen (fluoro, bromo, chloro, or iodo), nitro, trifluoromethyl, cyano, CO-alkyl, CO₂-alkyl, or CO₂-aryl. Chemical compounds such as carboxylic acids, carboxylic acid esters, nitrites, aromatic rings and ketones are all useful electron withdrawing groups.

Transition state analogs for prostasin can be easily designed and produced. Serine proteases typically have a similar active site geometry, such that hydrolysis of the substrate bond proceeds via the same mechanism of action. The first step in the reaction is the formation of an acyl-enzyme intermediate between the substrate and a conserved serine residue in the active site (hence the classification as a “serine protease”). The peptide bond is cleaved during formation of this covalent intermediate, which proceeds via a (negatively charged) tetrahedral transition state intermediate. Deacylation occurs during the second step of the mechanism of action, at which point the acyl-enzyme intermediate is hydrolyzed by a water molecule, the remaining portion of the substrate peptide is released, and the hydroxyl group of the serine residue is restored. The deacylation process also involves the formation of a tetrahedral transition state intermediate. As such, transition state analog compounds that mimic the structure of either of the tetrahedral intermediates can be employed as inhibitors of the serine protease.

Furthermore, chemical constituents that covalently modify or otherwise interact with the active site of the prostasin molecule can also be used as inhibitor moieties in the present invention. In some embodiments of the present invention, cleavage of the prostasin inhibitor molecule irreversibly deactivates the prostasin protease (e.g., a suicide inhibitor). In other embodiment, the inhibitor moiety need not be released from the prostasin inhibitor molecule to function as an inhibitor (e.g., an inhibitory affinity label). Optionally, the inhibitor moiety is activated upon release from the prostasin recognition site, and functions to either inhibit a single prostasin molecule or to catalyze the inhibition of a number of prostasin molecules. Mechanisms of serine protease inhibition are further described in Fersht (1985) Enzyme Structure and Mechanism (W.H. Freeman and Company, New York).

Typically, the peptide sequence in the prostasin inhibitors is typically based on the substrate specificity of prostasin. For example, the prostasin inhibitors can comprise a P₄-P₁ peptide sequence based on one or more of the prostasin substrates identified above, e.g., KHYR, RHYR, KHLR, KKLR, KHKR, RHFR, RKYR, KKYR, KHLR, RHLR, RKLR, KHKR, and RHFR. Some of the prostasin inhibitors can also comprise an acyl group at their P₄ residue. The transition state analog, mechanism-based moiety, or electron withdrawing moiety in the inhibitors can also comprise a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide, or the like. Thus, an exemplary prostasin inhibitor can comprise Acetyl-P₄-P₃-P₂-P₁-aldehylde, wherein P₄-P₁ comprises a non-prime substrate sequence as provided above.

The aldehyde inhibitor can be prepared using semicarbazone methodology. See, e.g., Dagino and Webb (1994) Tetrahedron Letters 35: 2125-2128. Dagino and Webb describe a method of making peptide aldehydes which involves using a diphenylmethyl semicarbazone group to provide a synthetic intermediate. For example, a protected diphenylmethyl semicarbazide derivative is synthesized, e.g., using techniques known to those of skill in the art. The semicarbazide is reacted to provide a protected argininal derivative, which is converted to a free amine, to which a desired peptide is linked, e.g., using standard peptide coupling techniques. The fully protected peptide aldehydes produced in this manner are optionally purified, e.g., using silica chromatography, and deprotected, e.g., by hydrogenation in acidic aqueous methanol.

VIII. Therapeutic Applications

Homologs of prostasin in other species have been shown to regulate ENaC activities. In a Xenopus kidney epithelial cell line, exposure of the apical membrane to the protease inhibitor aprotinin reduces transepithelial sodium transport (Vallet et al., Nature 389: 607-10 1997). Coexpression of the channel-activating protease, xCAP1, with ENaC in Xenopus oocytes increases the activity of the sodium channel by two- to threefold. Mouse membrane-bound channel-activating serine proteases (mCAP1, mCAP2, and mCAP3) were also able to activate ENaC in xenopus oocytes (Vuagniaux et al., J Gen Physiol 120: 191-201, 2002). Prostasin shares 41% and 76% sequence identity with xCAP1 and mCAP1, respectively. It was shown that co-expression of prostasin and ENaC in xenopus oocytes increased sodium transport by over 60% (Donaldson et al., J Biol Chem 277: 8338-45, 2002). These observations indicate that inhibition of prostasin activity could lead to inhibition of ENaC activation in the lung, thereby decreasing sodium hyperabsorption and mucociliary clearance in CF and possibly other lung disorders such as COPD and asthma.

The prostasin modulators (e.g., inhibitors) and prodrugs of the present invention are useful in the treatment of various diseases and conditions in which ENaC plays a role. Particularly amenable to treatment with compostions of the invention are diseases mediated by sodium hyperabsorption or abnormal mucociliary clearance in the lung. Examples of such diseases include CF, COPD, and asthma.

CF is the most common inherited, fatal disease in the world, affecting over 30,000 Americans alone. In cystic fibrosis, the mutant cystic fibrosis transmembrane conductance regulator (CFTR) results in increased ENaC activity and a depletion of ASL. As a result, the overlying mucus collapses onto the cilia and mucocillary clearance is disrupted leading to predisposition to bacterial infections (Pilewski and Frizzell 1999). A compound or prodrug that antagonizes the protease activity of prostasin would in turn inhibit ENaC activation. This could lead to a reversal of sodium hyperabsorption and ASL depletion.

Impaired mucociliary clearance has also been associated with chronic obstructive pulmonary disease (COPD). COPD is a disease that includes airflow obstruction that is associated with emphysema and chronic bronchitis. COPD and asthma affect more than 16 million and 10 million Americans, respectively. Despite several treatment options on the market, asthma and COPD still result in significant morbidity and mortality (5,000 and 120,000 deaths/year for asthma and COPD, respectively), showing the need for new treatments.

Prostasin inhibitors and prodrugs of the present invention are also suitable for treating other diseases and conditions in which such abnormal ENaC activity has been implicated. The prostasin inhibitors or prodrugs of the present invention can also be used in combination with any known drugs to treat subjects suffering from diseases or conditions wherein abnormal epithelial sodium channel plays a role. For example, a subject in need of treatment (e.g., of CF) can be administered a prostasin inhibitor of the present invention along with protease inhibitor aprotinin. In addition, there are also many known sodium channel blockers that can be employed in this aspect of the present invention. Pyrazinoylguanidine sodium channel blockers (e.g., amiloride, described in Merck Index), benzamil, and phenamil are examples of such known compounds. Additional sodium channel blockers are disclosed in, e.g., U.S. Pat. Nos. 3,313,813 and 6,479,498; and Kleyman et al., J. Membrane Biol. 105, 1-21, 1988.

The present invention also provides methods of inhibiting or reducing a prostasin activity in a cell. The methods involve contacting the cell with a prostasin inhibitor molecule containing a prostasin recognition site. Typically, the prostasin inhibitor molecule comprises a compound comprising the structure P₄P₃P₂P₁X, wherein P₁ is arginine; P₂ is tyrosine, leucine, phenylalanine, lysine, asparagine, or valine; P₃ is histidine, arginine, or lysine; and P₄ is arginine, lysine, histidine, tyrosine, proline, or leucine; and wherein X comprises an inhibitory moiety, such as a transition state analog, a mechanism-based inhibitor, or an electron withdrawing group. Exemplary inhibitory moieties include, but are not limited to, a C-terminal aldehyde, a boronate, a phosphonate, an α-ketoamide, a chloro methyl ketone, a sulfonyl chloride, ethyl propenoate, vinyl amide, vinyl sulfone, or vinyl sulfonamide.

To therapeutically or prophylactically treat a disease or disorder, one or more prostasin substrates, inhibitors or prodrugs of the present invention is administered to one or more cells. Typically, the prostasin inhibitors or prodrugs are administered in pharmaceutical compositions comprising a pharmaceutically acceptable excipient and one or more such prostasin substrates, inhibitors or prodrugs. In these in vivo methods, one or more cells of the subject, or a population of cells of interest, are contacted directly or indirectly with an amount of a prostasin substrate, inhibitor or prodrug composition of the present invention effective in prophylactically or therapeutically treating the disease, disorder, or other condition. In direct contact/administration formats, the composition is typically administered or transferred directly to the cells to be treated or to the tissue site of interest. In in vivo indirect contact/administration formats, the composition is typically administered or transferred indirectly to the cells to be treated or to the tissue site of interest (e.g., by the circulatory system or the lymph system).

Any of a variety of formats can be used to administer the compositions of the present invention (optionally along with one or more buffers and/or pharmaceutically-acceptable excipients), including inhaled administration, topical administration, transdermal administration, oral delivery, injection (e.g., by using a needle or syringe), placement within a cavity of the body (e.g., by catheter or during surgery), and the like. Pharmaceutically-acceptable excipients for use in the present invention include, but are not limited to, saline, buffered saline, dextrose, water, glycerol, ethanol, conventional nontoxic binders, disintegrants, flavorings, and carriers (e.g., pharmaceutical grades of mannitol, lactose, starch, magnesium stearate, sodium saccharine, talcum, cellulose, glucose, sucrose, magnesium, carbonate, and the like) and combinations thereof. The formulation is made to suit the mode of administration. Exemplary excipients and methods of formulation are provided, for example, in Remington's Pharmaceutical Science, 17th ed. (Mack Publishing Company, Easton, Pa., 1985).

Therapeutic compositions comprising one or more prostasin substrates, inhibitors or prodrugs of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

### EXAMPLES

The following examples are offered to illustrate, but not to limit the claimed invention.

### Example 1

### Cloning and Expression of Prostasin

Cloning:

The cDNA sequence of human prostasin (accession# NM_{—}002773) was isolated from a human testis cDNA library (Clontech) by PCR. The gene was modified by PCR to eliminate the N-terminal signal peptide domain and light chain and the C-terminal GPI signal domain. An N-terminal honeybee melittin signal peptide was added in the process. The final construct consisted of the 21 amino acid honeybee melittin signal peptide fused to Ile13 of prostasin. The final construct was subcloned into pFastBacl (Invitrogen).

Expression and Purification:

Protein expression was accomplished with the baculovirus system in Sf9 cells. Cells were cultured in InsectXpress serum-free medium (BioWhittaker) in vented shake flasks at 27C. Recombinant baculovirus was produced using the Bac-to-Bac expression system from Invitrogen. Cells in log phase were cultured to a density of 10⁶/ml and infected at an MOI of one. After 72 hours incubation, the supernatant was harvested. Detergent in the growth medium was precipitated by increasing the pH to 8.0 with 1M Tris-Cl pH 9.0. After centrifugation, the cleared medium was concentrated and diafiltrated against 50 mM sodium phosphate pH 7.4 by tangential flow. After adding NaCl to 300 mM and glycerol to 5%, the sample was passed over a cobalt IMAC resin (Clontech). The column was washed extensively and bound material was eluted with an imidazole gradient. Fractions were monitored for activity against a tetra peptide substrate (Ac-PRLR-acmc or Ac-KHYR-acmc). Active fractions were dialysed against 10 mM Hepes pH 7.4. Further purification was accomplished by size exclusion chromatography on Superdex 200 (Amersham).

Western Blot/Gel Stain:

Purified prostasin was visualized by silver stain and western blot. Electrophoresis and blotting materials were from Invitrogen. 0.5 μg prostasin in 1× LDS sample buffer and 100 mM β-mercaptoethanol was heated for 10 min at 70° C. and loaded in duplicate with 5 μl SeeBlue Plus2 standards onto a 10 well 4-12% Bis-tris polyacrylamide gel. After electrophoresis in MES buffer, the gel was cut in half. One half was blotted to PVDF in NuPage transfer buffer with 20% methanol while the other was stained with silver according to conventional methods. The western blot was probed with the India-His probe (Pierce) according to the manufacturer's instructions and developed with ECL+ (Amersham). The resulting signal as recorded with Kodak Biomax ML film indicates a high degree of purity of the purified prostasin.

### Example 2

### Characterization of Prostasin and Substrate Specificity

Enzyme Assays:

Proteolytic activity of the purified prostasin was monitored by measuring the accumulation of the fluorophore 7-amino-3-carbamoylmethyl-4-methylcoumarin (acmc) or 7-amino-4-carbamoylmethylcoumarin (acc) produced by the cleavage of the tetrapeptide substrate Ac-KHYR-acmc, Ac-KHYR-acc or Ac-PRLR-acmc in assay buffer (50 mM Tris-Cl pH 9.0, 0.25% CHAPS; 10 mM CHES pH 9.0 (37° C.), 0.25% CHAPS; or 10 mM Hepes pH 7.4 (37° C.), 0.25% CHAPS) at 37° C. Assays were run in 96 or 384-well black microtiter plates. Data was collected with a Gemini XS plate reader (Molecular Devices) at λₑₓ=380 nm and λₑₘ=450 nm.

Assay Buffer Optimization:

Prostasin activity was measured in the presence of detergents and other additives to find optimal assay buffer conditions. Various dilutions of 5%(w/v) CHAPS, 5% (v/v) Brij 35, 5% (v/v) Tween 20, 400 mM NaCl, 30% (v/v) glycerol, 100 mM EDTA, and 40 mM β-mercaptoethanol were diluted 2 fold in assay mixture (20 mM CHES pH 9.0 (37° C.), 50 nM prostasin, 100 μM Ac-PRLR-acmc). Accumulation of fluorophore was monitored at 37° C. as in “Enzyme Assays.”

Determination of Optimal Substrate

P₁-P₄-Fluorogenic substrate libraries were synthesized as previously described (Wang et al, J. Biol. Chem., 278: 15800-15808, 2003). Prostasin was diluted in assay buffer and added to the library plates. In the two-position fixed tetrapeptide substrate library, the final substrate concentration was approximately 0.25 μM/substrate/well with a total of 361 substrates per well. Each variable position had one of 19 amino acids with cysteine excluded and methionine replaced by its isostere norleucine. Accumulation of fluorophore was monitored as in “Enzyme Assays.” Results of the non-prime side positional scan are shown in FIGS. 1A-1C.

P₁′-P₄′-An optimal substrate sequence, KHYR (written N-terminus to C-terminus), was chosen based on the results of the P₁-P₄ libraries and used in the synthesis of a focused donor-quencher library for the elucidation of the P₁′-P₄′ substrate specificity of prostasin. An 80 well library was first synthesized using split and mix technology where each well contained a tetrapeptide sequence with one fixed and three variable amino acids followed by a lysine modified with a di-nitrophenyl quencher group and an arginine for enhanced solubility. Each fixed position was composed of one of 20 amino acids (excluding cysteine and including both methionine and its isostere norleucine). Following the P₁′ position of the hexapeptide library, the optimal non-prime sequence, RKFK, was synthesized and acylated with a fluorogenic coumarin donor group. After cleaving the substrates from a solid support, the library was lypophilized and dissolved in DMSO. For kinetic assays, 1 μl of the reconstituted library was added to 99 μl of HEPES-CHAPS buffer containing prostasin. The final concentration of each substrate in the assay was approximately 4 nM. The increase in fluorescence intensity was measured over time at λₑₓ=320 nm and λₑₘ=380 nm with a Gemini EM plate reader (Molecular Devices). FIGS. 2A-2D show results of the kinetic assays.

pH Profile

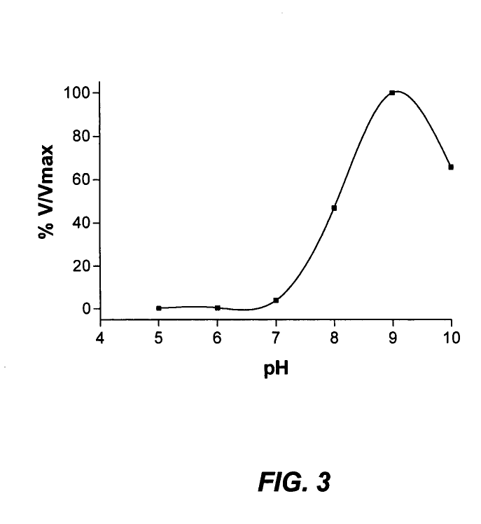

Prostasin was assayed in the following buffers in order to determine the pH where maximal activity occurs: 12.5 mM CAPS pH 10, 14.3 mM CHES pH 9.0, 13.9 mM Tris-Cl pH 8.0, 20 mM HEPES pH 7.0, 10.6 mM MES pH 6.0, 7.7 mM Acetate pH 5.0 (pHs at 37° C. and with 0.25% CHAPS). Buffer concentrations were calculated to give a total ionic strength of 5 mM. Activity against Ac-KHYR-acc was measured as in “Enzyme Assays.” The results are shown in FIG. 3.

Cation/Anion Inhibition

The inhibition constants of various cations and anions for prostasin were determined. Prostasin activity against Ac-KHYR-acc was measured in CHES-CHAPS buffer, as in “Enzyme Assays,” in the presence of 200 mM-0.2 mM NaCl, KCl, LiCl, and NaI, 250 mM-0.24 mM NaF, 250 mM-0.5 μM CaCl₂ and MgCl₂, and 100 μM-0.1 μM CoCl₂. Rates were plotted in Prism (GraphPad) and classical inhibition constants were calculated using the results of non-linear regression. Inhibition constants identified from this experiment is as follows: 5.3 mM (NaCl), 5.8 mM (KCl), 2.4 mM (LiCl), 4.5 mM (NaI), 4.2 mM (NaF), 0.26 mM (CaCl₂), 0.39 mM (MgCl₂), and 0.005 mM (CoCl₂).

Inhibition Assays

The inhibition constants of macromolecular inhibitors were calculated for prostasin. Prostasin activity against Ac-KHYR-acmc was measured in Tris-CHAPS buffer, as in “Enzyme Assays,” in the presence of 10 μM-170 pM aprotinin and soybean trypsin inhibitor (SBTI), 1 μM-17 pM α₁-antitrypsin, and 200 nM-3.3 pM α₁-antichymotrypsin. Rates were plotted in Prism (GraphPad) and classical inhibition constants (Kᵢ) were calculated using the results of non-linear regression. The results indicate that the classical inhibition constants of aprotinin, α₁-antitrypsin, α₁-antichymotrypsin, and SBTI on prostasin are 2.5 nM, >10 μM, >0.2 μM, and >10 μM, respectively.

### Example 3

### Metal Ions on Substrate-assisted Catalysis and Substrate Specificity

Assays with transition metals revealed substrate dependent, metal mediated effects that were not observed with metal ions from Groups I or II. In assays with Ac-KHYR-acmc, Kᵢs for prostasin increased by one to two orders of magnitude, and the order of metal inhibition potencies was altered. In the case of copper and zinc, an additional enhancement effect was observed where enzymatic activity increased up to 10 fold at sub inhibitory metal ion concentrations. An enhancement effect was also observed in assays with nickel, but in this case the activity increase was only 1.4 fold. No enhancement in activity was observed with cobalt. Cobalt, copper, nickel and zinc retained their inhibitory activity against prostasin, in assays with KHYR, but with 10, 25, 100 and 200 fold increases in IC50 values respectively and a change in relative inhibition potencies to Zn²⁺=Ni²⁺<Co²⁺<<Cu²⁺. These substrate dependent, metal ion mediated effects appeared to be unique to prostasin. Similar effects were not observed in identical assays with trypsin.

The effect of zinc on the Michelis-Menten kinetics of KHYR-acmc and PRLR-acmc was measured in the presence of 20 μM zinc(II). The addition of zinc resulted in a 4-fold decrease in the Km of KHYR-acmc and a 2 fold increase in the Km of PRLR-acmc. A similar but opposite effect was observed with k_{cat} values. The k_{cat} of KHYR-acmc was 2.7 fold higher and the k_{cat} of PRLR-acmc was 1.6 fold lower. These changes translated into a 30-fold increase in specificity for KHYR over PRLR in the presence of zinc.

Further, prostasin was assayed in the tetrapeptide substrate libraries with a set concentration of sodium, calcium or zinc to determine the effect of each on overall substrate specificity. Neither sodium nor calcium changed the substrate profile, but there was a decrease in activity across the libraries by 50-60% with sodium and 25-35% with calcium. The addition of zinc did not alter the specificity profiles generated from the P1 -P2, P1-P3, or P1-P4 libraries; the major specificity determinant remained in P1 with arginine and lysine preferred exclusively. In the P1-P3 library, however, there was a three fold relative increase in the activity of substrates with arginine in P1 and histidine in P3. This indicated a zinc dependent increase in selectivity for histidine in P3.

A zinc dependent increase in histidine selectivity in P3 was also reflected in the P2-P3 and P3-P4 libraries. In both libraries, most P3=H substrates were active. Without zinc, relatively few of these substrates were active in library P2-P3, and in library P3-P4, the substrates were mostly active but to a lesser extent. Notable among inactive P3=H substrates were P2=Y,W in library P2-P3 and P4=H in library P3-P4. In library P2-P4, selectivity for His in P4 emerged as well but was weaker than the zinc dependent selectivity for histidine in P3 seen in the other libraries. Interestingly, zinc dependent selectivity for histidine in P3 and P4 appeared to be mutually exclusive: increased specificity was not observed with substrates that had histidine in both positions.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, patents, patent applications, and/or other documents cited in this application are incorporated by reference in their entirety and for all purposes to the same extent as if each individual publication, patent, patent application, or other document were individually so denoted.

1. A prostasin-cleavable molecule that comprises a prostasin cleavage site, wherein the prostasin-cleavable molecule comprises:
P₄P₃P₂P₁X
wherein:
P₁ is arginine;
P₂ is tyrosine, leucine, phenylalanine, lysine, asparagine, or valine;
P₃ is histidine, arginine, or lysine;
P₄ is arginine, lysine, histidine, tyrosine, proline, or leucine; and
X comprises one or more of an inhibitory moiety, a label moiety, a polypeptide comprising 1 to 25 amino acids, or a polypeptide that is not attached to P₄P₃P₂P₁ in a naturally occurring protein; and
wherein the prostasin cleavage site is between P₁ and X.

2. The prostasin-cleavable molecule of claim 1, wherein P₁ is arginine, P₂ is tyrosine or leucine, P₃ is histidine, lysine, or arginine, and P₄ is lysine or arginine.

3. The prostasin-cleavable molecule of claim 1, wherein P₄P₃P₂P₁ has a sequence selected from the group consisting of KHYR, RHYR, KHLR, KKLR, KHKR, RKYR, KKYR, KHLR, RHLR, RKLR, and KHKR.

4. The prostasin-cleavable molecule of claim 1, wherein P₄P₃P₂P₁ has a sequence of KHYR.

5. The prostasin-cleavable molecule of claim 1, wherein X comprises P₁′P₂′P₃′P₄′, wherein: P₁′ is attached to P₁ and is histidine, arginine, lysine, asparagine, glutamine, serine, norleucine, or alanine; P₂′ is proline, alanine, histidine, asparagine, norleucine, or glutamine; P₃′ is histidine, serine, glutamine, or aspartic acid; and P₄′ is alanine, serine, norleucine, asparagine, leucine, or threonine.

6. The prostasin-cleavable molecule of claim 1, wherein the label moiety comprises an absorbent, fluorescent or luminescent label moiety.

7. The prostasin-cleavable molecule of claim 6, wherein the label moiety comprises a fluorophore, a coumarin moiety, or a rhodamine moiety.

8. The prostasin-cleavable molecule of claim 7, wherein the coumarin moiety comprises 7-amino-4-carbamoylcoumarin, 7-amino-3-carbamoylmethyl-4-methylcoumarin, or 7-amino-4-methylcoumarin.

9. The prostasin-cleavable molecule of claim 6, wherein the prostasin-cleavable molecule comprises a first member of a fluorescence resonance transfer energy pair attached to the molecule on one side of the prostasin cleavage site and a second member of the fluorescence resonance transfer energy pair attached to the molecule on the opposite side of the prostasin cleavage site.

10. The prostasin-cleavable molecule of claim 9, wherein the fluorescence resonance transfer energy pair comprises amino benzoic acid and nitro-tyrosine; 7-methoxy-3-carbamoyl-4-methylcoumarin and dinitrophenol; or 7-dimethylamino-3-carbamoyl-4-methylcoumarin and dabsyl.

11. A prostasin-cleavable peptide that comprises fewer than 25 amino acids, the peptide comprising P₄P₃P₂P₁, wherein P₁ is arginine or lysine; P₂ is tyrosine, leucine, phenylalanine, lysine, asparagine, or valine; P₃ is histidine, arginine, or lysine; and P₄ is arginine, lysine, histidine, tyrosine, proline, or leucine; and one or more amino acids attached to either or both of P₁ and P₄.

12. The prostasin-cleavable peptide of claim 11, wherein P₁ is arginine, P₂ is tyrosine or leucine, P₃ is histidine, lysine, or arginine, and P₄ is lysine or arginine.

13. The prostasin-cleavable peptide of claim 11, the peptide further comprising 1 to 20 amino acids linked to P₄.

14. The prostasin-cleavable peptide of claim 11, the peptide further comprising 1 to 20 amino acids linked to P₁.

15. The prostasin-cleavable peptide of claim 11, the peptide further comprising P₁′P₂′P₃′P₄′, wherein P₁′ is attached to P₁ and is histidine, arginine, lysine, asparagine, glutamine, serine, norleucine, or alanine; P₂′ is proline, alanine, histidine, asparagine, norleucine, or glutamine; P₃′ is histidine, serine, glutamine, or aspartic acid; and P₄′ is alanine, serine, norleucine, asparagine, leucine, or threonine.

16. A prostasin inhibitor comprising P₄P₃P₂P₁Z, wherein P₁ comprises arginine or lysine; P₂ comprises tyrosine, leucine, phenylalanine, lysine, asparagine, or valine; P₃ comprises histidine, arginine, or lysine; and P₄ comprises arginine, lysine, histidine, tyrosine, proline, or leucine; and Z comprises a transition state analog, a mechanism-based inhibitor, or an electron withdrawing group.

17. The prostasin inhibitor of claim 16, wherein P₁ is arginine, P₂ is tyrosine or leucine, P₃ is histidine, lysine, or arginine, and P₄ is lysine or arginine.

18. The prostasin inhibitor of claim 16, wherein P₄ comprises acetyl-lysine.

19. The prostasin inhibitor of claim 16, wherein the transition state analog, mechanism-based inhibitor, or electron withdrawing moiety comprises a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide.

20. A method for identifying a modulator of prostasin, the methods comprising (a) contacting a test agent with prostasin in the presence of a prostasin substrate, and (b) detecting an alteration of cleavage of the prostasin substrate by prostasin in the presence of the test agent relative to cleavage of the prostasin substrate by prostasin in the absence of the test agent; thereby identifying a prostasin modulator; and
wherein the prostasin substrate comprises P₄P₃P₂P₁X, wherein P₁ is arginine; P₂ is tyrosine, leucine, phenylalanine, lysine, asparagine, or valine; P₃ is histidine, arginine, or lysine; and P₄ is arginine, lysine, histidine, tyrosine, proline, or leucine; and X comprises a polypeptide that comprises 1 to 25 amino acids and that is not attached to P₄P₃P₂P₁ in a naturally occurring protein.

21. The method of claim 20, wherein P₁ is arginine, P₂ is tyrosine or leucine, P₃ is histidine, lysine, or arginine, and P₄ is lysine or arginine.

22. The method of claim 20, wherein X comprises P₁′P₂′P₃′P₄′, wherein P₁′ is attached to P₁ and is histidine, arginine, lysine, asparagine, glutamine, serine, norleucine, or alanine; P₂′ is proline, alanine, histidine, asparagine, norleucine, or glutamine; P₃′ is histidine, serine, glutamine, or aspartic acid; and P₄′ is alanine, serine, norleucine, asparagine, leucine, or threonine.

23. The method of claim 22, wherein the prostasin substrate further comprises a fluorescence resonance energy transfer pair having a first member coupled to the one or more prime positions and a second member coupled to the one or more non-prime positions.

24. The method of claim 23, wherein the fluorescence resonance transfer energy pair comprises amino benzoic acid and nitro-tyrosine; 7-methoxy-3-carbamoyl-4-methylcoumarin and dinitrophenol, or 7-dimethylamino-3-carbamoyl-4-methylcoumarin and dabsyl.

25. The method of claim 23, wherein alteration of cleavage of the substrate by prostasin is detected by monitoring fluorescence resonance energy transfer between the members of the fluorescence resonance energy transfer pair.

26. A method of reducing a prostasin activity in a cell, the method comprising contacting the cell with a prostasin inhibitor molecule, wherein the prostasin inhibitor molecule comprises P₄P₃P₂P₁Z, wherein P₁ comprises arginine; P₂ comprises tyrosine, leucine, phenylalanine, lysine, asparagine, or valine; P₃ comprises histidine, arginine, or lysine; and P₄ comprises arginine, lysine, histidine, tyrosine, proline, or leucine; and Z comprises an inhibitory moiety.

27. The method of claim 26, wherein the inhibitory moiety is a transition state analog, a mechanism-based inhibitor, or an electron withdrawing group.

28. The method of claim 26, wherein the cell is in a mammal.

29. The method of claim 26, wherein the cell is in a human.

30. The method of claim 26, wherein the prostasin inhibitor is applied to the cell in a pharmaceutically acceptable excipient.
